(19) United States
(12) Patent Application Publication
(10) Pub. No.: US 2002/0091082 A1
(43) Pub. Date: Jul. 11, 2002

(54) Methods of modulating symptoms of hypertension

(76) Inventor: Lloyd P. Aiello, Cambridge, MA (US)

Correspondence Address: LOUIS MYERS, Fish & Richardson P.C., 225 Franklin Street, Boston, MA, 02110-2804, US

(21) Appl. No.: 09/952,350

(22) Filed: Sep. 13, 2001

Related U.S. Application Data
(60) Provisional application No. 60/232,503, filed on Sep. 13, 2000.

(51) Int. Cl. A61K 48/00; A61K 38/17; A61K 39/395

(52) U.S. Cl. 514/12; 514/44; 424/146.1

(57) ABSTRACT

The invention features methods of treating hypertension and related disorders and conditions, e.g., diabetic retinopathy, by inhibiting VEGF-KDR signaling pathway components, e.g., PKC-zeta and/or PI3 kinase 1.

### RELATED APPLICATION

This application claims the benefit of U.S. Provisional Application Serial No. 60/232,503, filed Sep. 13, 2000, which is incorporated herein by reference in its entirety.

### FEDERALLY SPONSORED RESEARCH OR DEVELOPMENT

The U.S. Government may have certain rights in this invention pursuant to Grant No. EY10827 awarded by National Eye Institute, National Institutes of Health.

### BACKGROUND

Concomitant hypertension exacerbates a wide variety of disease including ocular disorders such as diabetic retinopathy, age related macular degeneration, retinal vein inclusion, and retinal macro aneurysms. In addition, hypertension itself causes a significant retinopathy as well as alterations throughout the body including increased risk of cardiovascular disease, myocardial infraction, stroke and death. The mechanisms by which hypertension exacerbates these assorted disorders are not well understood.

Numerous vision-threatening diseases such as diabetic retinopathy are exacerbated by coexistent hypertension. Epidemiological studies identify hypertension as an independent risk factor for diabetic retinopathy. Patients with higher ranges of blood pressure are up to three times more likely to develop PDR diabetic retinopathy (Roy (2000) Arch. Ophthalmol. 118: 105-115), 35% more likely to have retinopathy progression, 47% more likely to have visual loss (UK Prospective Diabetes Study Group. (1998) British Medical Journal 317: 703-713) and three times more likely to develop diffuse macular edema (Lopes et al. (1999) Acta Ophthalmol Scand. 77: 170-175). The sight threatening complications of diabetic retinopathy are characterized by development of retinal neovascularization and/or retinal vascular permeability. Severe hypertension itself can induce a retinopathy characterized by increased retinal vascular leakage.

Hypertension increases large artery and retinal artery dilation much as 15% (Safar et al. (1981) Circulation 63: 393-400) and 35%, (Houben et al.(1995) J. Hypertens. 13: 1729-1733) respectively. Mechanical stretch can initiate intracellular signaling, regulate protein synthesis and alter secretion of numerous factors including NO, endothelin-1, platelet-derived growth factor, fibroblast growth factor and angiotensin II. Recently, mechanical stretch has been shown to induce expression in rat ventricular myocardium, myocytes and human mesangial cells. (Li J et al. (1997), J Clin. Invest. 100: 18-24; Seko Y et al.(1999) Biochem. Biophys. Res. Commun. 254: 462-465; Gruden G, et al.,(1997) Proc. Natl. Acad. Sci. U. S. A. 94: 12112-12116).

### SUMMARY OF THE INVENTION

This invention is based, in part, on the following discoveries: (1) the discovery that the additional stretch experienced by the vasculature during hypertension increases the expression and activity of vascular endothelial growth factor (VEGF) and its receptor KDR; (2) the identification that the aforementioned increases in VEGF and KDR are mediated by PI3 kinase and by a specific isoform of protein kinase C, namely the zeta(ζ) isoform of PKC (PKC-zeta). These findings provide methods of preventing hypertension by modulating, e.g., inhibiting VEGF, the VEGF receptor KDR, PI3 kinase and PKC-zeta. Thus, the invention can be used to treat hypertension and related disorders and diseases, e.g., disorders exacerbated by hypertension, e.g., hypertensive retinopathy, age-related macular degeneration (AMD) or diabetic retinopathy.

Accordingly, one aspect of the invention features a method of treating a vascular disorder. The method includes administering to a subject an effective dose of an agent which modulates, preferably reduces, VEGF-KDR signaling. For example, VEGF-KDR signaling can be reduced by reducing, e.g., KDR expression, KDR protein levels, and/or_KDR activity; reducing VEGF expression, levels, or activity; reducing PI3 kinase expression, levels, or activity; and/or reducing PKC-zeta expression, levels, or activity.

In a preferred embodiment, VEGF-KDR signaling is reduced in vivo in the subject.

In another preferred embodiment, VEGF-KDR signaling is reduced ex-vivo in a cell or tissue of the subject, and the cell or tissue is transplanted back into the subject. The cell or tissue of the subject can be an endothelial cell, e.g., a cardiac endothelial cell or a retinal endothelial cell or a pericyte. In another embodiment, the agent reduces VEGF-KDR signaling in a cell or tissue subjected to a mechanical stress. In one embodiment, the cell is subject to mechanical stress, e.g., as a result of an increase in vascular pressure, e.g., increased blood pressure or hypertension.

The subject can be a human or a non-human animal, e.g., an experimental animal. The experimental animal can be a rat, preferably a spontaneous hypertensive rat. In one embodiment, a cell or tissue in the experimental animal is subjected, in vivo or ex vivo, to mechanical stress as result of mechanical strain from a device, e.g., a vacuum stretch apparatus. In a preferred embodiment, the strain is produced with a cardiac profile, e.g., with a frequency of about 60 cpm. The strain can uniform in radial and circumferential dimensions. In one embodiment, the cell is an endothelial cell, e.g., a bovine retinal microvascular endothelial cell (BREC); or a pericyte.

In one embodiment, the vascular disorder is hypertension, e.g., concomitant hypertension. In one embodiment, hypertension exacerbates an ocular disorder such as diabetic retinopathy, hypertensive retinopathy, age-related macular degeneration (AMD), retinal vein inclusion, or retinal macro aneurysms. In a preferred embodiment, hypertension exacerbates diabetic retinopathy. In another embodiment, hypertension exacerbates a cardiovascular disease, e.g., myocardial infarction or stroke.

In a preferred embodiment, hypertension is modulated in a subject that is at risk for a hypertension-related disorder, e.g., the subject has diabetes and is administered an agent that modulates VEGF-KDR signaling, e.g., in order to treat or prevent the hypertension related disorder. Optionally, the method includes identifying a subject who is at risk for hypertension or a hypertension related disorder, e.g., identifying a subject who has diabetes. Examples of hypertension-related disorders include, but are not limited to, retinal disorders such as diabetic retinopathy, hypertensive retinopathy, age-related macular degeneration (AMD), retinal vein inclusion, or retinal macro aneurysms.

In one preferred embodiment, VEGF-KDR signaling is inhibited by reducing VEGF expression, levels, or activity, e.g., by administering an agent that reduces VEGF expression levels, or activity. VEGF can be inhibited by administering an agent which inhibits VEGF gene expression, protein production levels and/or activity. An agent which inhibits VEGF can be one or more of: a VEGF binding protein, e.g., a soluble VEGF binding protein, e.g., the ectodomain of a VEGF-receptor; an antibody that specifically binds to the VEGF protein, e.g., an antibody that disrupts VEGF's ability to bind to its natural cellular target, e.g., disrupts VEGF's ability to bind to a VEGF receptor, e.g., KDR; an antibody that disrupts the ability of a VEGF receptor, e.g., KDR, to bind to VEGF; an antibody or small molecule which disrupts a complex formed by VEGF and KDR; a mutated inactive VEGF or fragment which binds to KDR but does not activate the receptor; a VEGF nucleic acid molecule which can bind to a cellular VEGF nucleic acid sequence, e.g., mRNA, and inhibit expression of the protein, e.g., an antisense molecule or VEGF ribozyme; an agent which decreases VEGF gene expression, e.g., a small molecule which binds the promoter of VEGF. In another preferred embodiment, VEGF is inhibited by decreasing the level of expression of an endogenous VEGF gene, e.g., by decreasing transcription of the VEGF gene. In a preferred embodiment, transcription of the VEGF gene can be decreased by: altering the regulatory sequences of the endogenous VEGF gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, the antibody which binds VEGF is a monoclonal antibody, e.g., a humanized chimeric or human monoclonal antibody.

In another preferred embodiment, VEGF-KDR signaling is inhibited by administering an agent that inhibits KDR expression, levels, or activity. An agent which inhibits KDR can be one or more of: a KDR binding protein, e.g., a KDR binding protein that binds to KDR but does not activate KDR, e.g., a portion of VEGF which binds to KDR but does not activate KDR; an antibody which binds KDR; an agent which decreases KDR gene expression, e.g., a KDR nucleic acid molecule which can bind to a cellular KDR nucleic acid sequence, e.g., mRNA, and inhibit expression of the protein, e.g., an antisense molecule or KDR ribozyme; an agent which decreases KDR gene expression, e.g., a small molecule which binds the promoter of KDR. In another preferred embodiment, KDR is inhibited by decreasing the level of expression of an endogenous KDR gene, e.g., by decreasing transcription of the KDR gene. In a preferred embodiment, transcription of the KDR gene can be decreased by: altering the regulatory sequences of the endogenous KDR gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, the antibody which binds KDR is a monoclonal antibody, e.g., a human or humanized monoclonal antibody.

In another preferred embodiment, VEGF-KDR signaling is inhibited by reducing PI3 kinase expression, levels, or activity, e.g., by administering an agent that reduces PI3 kinase expression, levels, or activity. An agent which inhibits PI3-kinase activity can be one or more of: a small molecule which inhibits PI3-kinase activity, e.g., LY294002 or wortmannin; a protein or peptide that inhibits PI3 kinase activity, e.g., a PI3 kinase binding protein which binds to PI3-kinase but does not activate the enzyme, or a dominant negative form of p85; an antibody that specifically binds to the PI3-kinase protein, e.g., an antibody that disrupts PI3-kinase's catalytic activity or an antibody that disrupts the ability of cellular receptors to activate PI3-kinase; a PI3 kinase nucleic acid molecule which can bind to a cellular PI3 kinase nucleic acid sequence, e.g., mRNA, and inhibit expression of the protein, e.g., an antisense molecule or PI3-kinase ribozyme; an agent which decreases PI3-kinase gene expression, e.g., a small molecule which binds the promoter of PI3-kinase. In another preferred embodiment, PI3-kinase is inhibited by decreasing the level of expression of an endogenous PI3-kinase gene, e.g., by decreasing transcription of the PI3-kinase gene. In a preferred embodiment, transcription of the PI3-kinase gene can be decreased by: altering the regulatory sequences of the endogenous PI3-kinase gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, PI3-kinase activity is inhibited by a small molecule inhibitor, e.g., wortmannin or LY294002.

In another preferred embodiment, VEGF-KDR signaling is inhibited by inhibiting PKC, preferably PKC-zeta expression, levels, or activity, e.g., by administering an agent that inhibits PKC-zeta. An agent which inhibits PKC activity, e.g., PKC-zeta activity, can be one or more of: a small molecule which inhibits PKC expression or activity, e.g., PKC-zeta expression or activity; a PKC binding protein which binds to PKC, e.g., PKC-zeta, but does not activate the enzyme; an antibody that specifically binds to the PKC protein, e.g., PKC-zeta, e.g., an antibody that disrupts PKC-zeta catalytic activity or an antibody that disrupts the ability of upstream activators to activate PKC-zeta; a PKC nucleic acid molecule which can bind to a cellular PKC nucleic acid sequence, e.g., mRNA, e.g., PKC-zeta MRNA, and inhibit expression of the protein, e.g., an antisense molecule or PKC ribozyme; an agent which decreases PKC-zeta gene expression, e.g., a small molecule which binds the promoter of PKC-zeta. In another preferred embodiment, PKC, e.g., PKC-zeta is inhibited by decreasing the level of expression of an endogenous PKC gene, e.g., by decreasing transcription of the PKC-zeta gene. In a preferred embodiment, transcription of the PKC-zeta gene can be decreased by: altering the regulatory sequences of the endogenous PKC-zeta gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, PKC-zeta activity is inhibited by a specific small molecule inhibitor. In another preferred embodiment, PKC-zeta activity is inhibited by a monoclonal antibody, e.g., a human or humanized monoclonal antibody.

In another aspect, the invention features a method of modulating hypertension and/or mechanical stress in a cell, tissue, or subject. The method includes administering to a subject an effective dose of an agent which modulates, preferably reduces, VEGF-KDR signaling. For example, VEGF-KDR signaling can be reduced by reducing, e.g., KDR expression, KDR protein levels, and/or_KDR activity; reducing VEGF expression, levels, or activity; reducing PI3 kinase expression, levels, or activity; and/or reducing PKC-zeta expression, levels, or activity.

In a preferred embodiment, VEGF-KDR signaling is reduced in vivo in the subject.

In another preferred embodiment, VEGF-KDR signaling is reduced ex-vivo in a cell or tissue of the subject, and the cell or tissue is transplanted back into the subject. The cell or tissue of the subject can be an endothelial cell, e.g., a cardiac endothelial cell or a retinal endothelial cell or a pericyte. In another embodiment, the agent reduces VEGF-KDR signaling in a cell or tissue subjected to a mechanical stress. In one embodiment, the cell is subject to mechanical stress, e.g., as a result of an increase in vascular pressure, e.g., increased blood pressure or hypertension.

The subject can be a human or a non-human animal, e.g., an experimental animal. The experimental animal can be a rat, preferably a spontaneous hypertensive rat. In one embodiment, a cell or tissue in the experimental animal is subjected, in vivo or ex vivo, to mechanical stress as result of mechanical strain from a device, e.g., a vacuum stretch apparatus. In a preferred embodiment, the strain is produced with a cardiac profile, e.g., with a frequency of about 60 cpm. The strain can uniform in radial and circumferential dimensions. In one embodiment, the cell is an endothelial cell, e.g., a bovine retinal microvascular endothelial cell (BREC); or a pericyte.

In one embodiment, the hypertension is, e.g., concomitant hypertension. In one embodiment, hypertension exacerbates an ocular disorder such as diabetic retinopathy, hypertensive retinopathy, age-related macular degeneration (AMD), retinal vein inclusion, or retinal macro aneurysms. In a preferred embodiment, hypertension exacerbates diabetic retinopathy. In another embodiment, hypertension exacerbates a cardiovascular disease, e.g., myocardial infarction or stroke.

In a preferred embodiment, hypertension is modulated in a subject that is at risk for a hypertension-related disorder, e.g., the subject has diabetes and is administered an agent that modulates VEGF-KDR signaling, e.g., in order to treat or prevent the hypertension related disorder. Optionally, the method includes identifying a subject who is at risk for hypertension or a hypertension related disorder, e.g., identifying a subject who has diabetes. Examples of hypertension-related disorders include, but are not limited to, retinal disorders such as diabetic retinopathy, hypertensive retinopathy, age-related macular degeneration (AMD), retinal vein inclusion, or retinal macro aneurysms.

In one preferred embodiment, VEGF-KDR signaling is inhibited by reducing VEGF expression, levels, or activity, e.g., by administering an agent that reduces VEGF expression levels, or activity. VEGF can be inhibited by administering an agent which inhibits VEGF gene expression, protein production levels and/or activity. An agent which inhibits VEGF can be one or more of: a VEGF binding protein, e.g., a soluble VEGF binding protein, e.g., the ectodomain of a VEGF-receptor; an antibody that specifically binds to the VEGF protein, e.g., an antibody that disrupts VEGF's ability to bind to its natural cellular target, e.g., disrupts VEGF's ability to bind to a VEGF receptor, e.g., KDR; an antibody that disrupts the ability of a VEGF receptor, e.g., KDR, to bind to VEGF; an antibody or small molecule which disrupts a complex formed by VEGF and KDR; a mutated inactive VEGF or fragment which binds to KDR but does not activate the receptor; a VEGF nucleic acid molecule which can bind to a cellular VEGF nucleic acid sequence, e.g., mRNA, and inhibit expression of the protein, e.g., an antisense molecule or VEGF ribozyme; an agent which decreases VEGF gene expression, e.g., a small molecule which binds the promoter of VEGF. In another preferred embodiment, VEGF is inhibited by decreasing the level of expression of an endogenous VEGF gene, e.g., by decreasing transcription of the VEGF gene. In a preferred embodiment, transcription of the VEGF gene can be decreased by: altering the regulatory sequences of the endogenous VEGF gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, the antibody which binds VEGF is a monoclonal antibody, e.g., a humanized chimeric or human monoclonal antibody.

In another preferred embodiment, VEGF-KDR signaling is inhibited by administering an agent that inhibits KDR expression, levels, or activity. An agent which inhibits KDR can be one or more of: a KDR binding protein, e.g., a KDR binding protein that binds to KDR but does not activate KDR, e.g., a portion of VEGF which binds to KDR but does not activate KDR; an antibody which binds KDR; an agent which decreases KDR gene expression, e.g., a KDR nucleic acid molecule which can bind to a cellular KDR nucleic acid sequence, e.g., mRNA, and inhibit expression of the protein, e.g., an antisense molecule or KDR ribozyme; an agent which decreases KDR gene expression, e.g., a small molecule which binds the promoter of KDR. In another preferred embodiment, KDR is inhibited by decreasing the level of expression of an endogenous KDR gene, e.g., by decreasing transcription of the KDR gene. In a preferred embodiment, transcription of the KDR gene can be decreased by: altering the regulatory sequences of the endogenous KDR gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, the antibody which binds KDR is a monoclonal antibody, e.g., a human or humanized monoclonal antibody.

In another preferred embodiment, VEGF-KDR signaling is inhibited by reducing PI3 kinase expression, levels, or activity, e.g., by administering an agent that reduces PI3 kinase expression, levels, or activity. An agent which inhibits PI3-kinase activity can be one or more of: a small molecule which inhibits PI3-kinase activity, e.g., LY294002 or wortmannin; a protein or peptide that inhibits PI3 kinase activity, e.g., a PI3 kinase binding protein which binds to PI3-kinase but does not activate the enzyme, or a dominant negative form of p85; an antibody that specifically binds to the PI3-kinase protein, e.g., an antibody that disrupts PI3-kinase's catalytic activity or an antibody that disrupts the ability of cellular receptors to activate PI3-kinase; a PI3 kinase nucleic acid molecule which can bind to a cellular PI3 kinase nucleic acid sequence, e.g., mRNA, and inhibit expression of the protein, e.g., an antisense molecule or PI3-kinase ribozyme; an agent which decreases PI3-kinase gene expression, e.g., a small molecule which binds the promoter of PI3-kinase. In another preferred embodiment, PI3-kinase is inhibited by decreasing the level of expression of an endogenous PI3-kinase gene, e.g., by decreasing transcription of the PI3-kinase gene. In a preferred embodiment, transcription of the PI3-kinase gene can be decreased by: altering the regulatory sequences of the endogenous PI3-kinase gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, PI3-kinase activity is inhibited by a small molecule inhibitor, e.g., wortmannin or LY294002.

In another preferred embodiment, VEGF-KDR signaling is inhibited by inhibiting PKC, preferably PKC-zeta expression, levels, or activity, e.g., by administering an agent that inhibits PKC-zeta. An agent which inhibits PKC activity, e.g., PKC-zeta activity, can be one or more of: a small molecule which inhibits PKC expression or activity, e.g., PKC-zeta expression or activity; a PKC binding protein which binds to PKC, e.g., PKC-zeta, but does not activate the enzyme; an antibody that specifically binds to the PKC protein, e.g., PKC-zeta, e.g., an antibody that disrupts PKC-zeta catalytic activity or an antibody that disrupts the ability of upstream activators to activate PKC-zeta; a PKC nucleic acid molecule which can bind to a cellular PKC nucleic acid sequence, e.g., mRNA, e.g., PKC-zeta mRNA, and inhibit expression of the protein, e.g., an antisense molecule or PKC ribozyme; an agent which decreases PKC-zeta gene expression, e.g., a small molecule which binds the promoter of PKC-zeta. In another preferred embodiment, PKC, e.g., PKC-zeta is inhibited by decreasing the level of expression of an endogenous PKC gene, e.g., by decreasing transcription of the PKC-zeta gene. In a preferred embodiment, transcription of the PKC-zeta gene can be decreased by: altering the regulatory sequences of the endogenous PKC-zeta gene, e.g., by the addition of a negative regulatory sequence (such as a DNA-biding site for a transcriptional repressor), or by the removal of a positive regulatory sequence (such as an enhancer or a DNA-binding site for a transcriptional activator). In another preferred embodiment, PKC-zeta activity is inhibited by a specific small molecule inhibitor. In another preferred embodiment, PKC-zeta activity is inhibited by a monoclonal antibody, e.g., a human or humanized monoclonal antibody.

In another aspect, the invention features a method of treating a symptom of a vascular disorder, e.g., hypertension. The method includes administering to a subject an effective dose of an agent described hereinabove which reduces VEGF-KDR signaling to thereby treat the disorder. In one embodiment, the vascular disorder is hypertension, e.g., concomitant hypertension. In one embodiment, hypertension exacerbates an ocular disorder such as diabetic retinopathy, age-related macular degeneration, retinal vein inclusion, retinal macro aneurysms, etc. In a preferred embodiment, hypertension exacerbates diabetic retinopathy. In another embodiment, hypertension exacerbates a cardiovascular disease, e.g., myocardial infarction and stroke.

In a preferred embodiment, hypertension is modulated in a subject that is at risk for a hypertension-related disorder, e.g., the subject has diabetes and is administered an agent that modulates VEGF-KDR signaling, e.g., in order to treat or prevent the hypertension related disorder. Optionally, the method includes identifying a subject who is at risk for hypertension or a hypertension related disorder, e.g., identifying a subject who has diabetes. Examples of hypertension-related disorders include, but are not limited to, retinal disorders such as diabetic retinopathy, hypertensive retinopathy, age-related macular degeneration (AMD), retinal vein inclusion, or retinal macro aneurysms.

Another aspect of the invention features a method of screening for a test compound which reduces a symptom of hypertension. The method includes providing an endothelial cell or tissue, e.g., a retinal endothelial cell or tissue; contacting the cell or tissue with a test compound; and evaluating the VEGF-KDR signaling to thereby identify a test compound which ameliorates a symptom of hypertension. A reduction in VEGF-KDR signaling in the test cell or tissue compared to a control is indicative of an agent which reduces a symptom of hypertension. In one embodiment, the method also includes subjecting the cell to a mechanical stress, e.g., stretch. A reduction in the mechanical stress-induced VEGF-KDR signaling relative to the increase in a test cell or tissue not contacted with the test agent is indicative of an agent which reduces a symptom of hypertension. In preferred embodiments, the screening can include screening for: an agent that inhibits VEGF activity; an agent that inhibits KDR signaling, e.g., an agent that inhibits the interaction between VEGF and a KDR; an agent that inhibits PI3 kinase activity; an agent that inhibits a KDR interaction with p85 subunit of PI3-kinase; an agent that inhibits PKC-zeta activity.

In one embodiment, the endothelial cell is a cardiac endothelial cell, a retinal endothelial cell, or a pericyte. In one preferred embodiment, the cell is a bovine retinal microvascular endothelial cell (BREC). In another preferred embodiment, the cell is a retinal pericyte. In one embodiment, the cell is subject to mechanical stress as a result of an increase in vascular pressure, e.g., increased blood pressure or hypertension. The cell can be in a live animal, e.g., an experimental model, e.g., a rat, a mouse, a non-human primate, a pig, a dog, or a cat. In one embodiment, the experimental animal is a rat, e.g., a spontaneous hypertensive rat. In another embodiment, the animal is a transgenic animal. In another embodiment, the cell is subjected to mechanical stress as result of mechanical strain from a device, e.g., a vacuum stretch apparatus. In a preferred embodiment, the strain is produced with a cardiac profile, e.g., with a frequency of about 60 cpm. The strain can uniform in radial and circumferential dimensions.

In a preferred embodiment, the method further includes administering the test agent to an experimental animal.

VEGF, KDR, PI3 kinase and/or PKC-zeta expression, levels or activity can be assayed by various methods commonly practiced in the art. In one embodiment, VEGF, KDR, PI3 kinase and/or PKC-zeta expression levels are assayed by Northern analysis. In another embodiment VEGF, KDR, PI3 kinase and/or PKC protein levels are assayed by detecting protein with an antibody, e.g., using an ELISA assay or a Western blot assay. In other embodiments, standard PKC and/or PI3 kinase assays can be used in the evaluating step of the screening assays described herein.

In another aspect, the invention features diagnostic methods to determine if a subject is at risk for hypertension or a related disorder or condition, e.g., a retinal disorder, e.g., retinopathy. The methods include one or more of:

detecting, in a tissue, e.g., an endothelial tissue, of the subject, the presence or absence of a mutation which affects the expression of a gene involved in VEGF-KDR signaling (e.g., VEGF, KDR, PI3 kinase, PKC-zeta), or detecting the presence or absence of a mutation in a region which controls the expression of the gene, e.g., a mutation in the 5′ control region;

detecting, in a tissue of the subject, the presence or absence of a mutation which alters the structure of a gene gene involved in VEGF-KDR signaling (e.g., VEGF, KDR, PI3 kinase, PKC-zeta);

detecting, in a tissue of the subject, the misexpression of a gene involved in VEGF-KDR signaling (e.g., VEGF, KDR, PI3 kinase, PKC-zeta), at the mRNA level, e.g., detecting a non-wild type level of a mRNA;

detecting, in a tissue of the subject, the misexpression of a gene involved in VEGF-KDR signaling (e.g., VEGF, KDR, PI3 kinase, PKC-zeta), at the protein level, e.g., detecting a non-wild type level of a protein encoded by the gene.

In preferred embodiments the method includes: ascertaining the existence of at least one of: a deletion of one or more nucleotides from the gene; an insertion of one or more nucleotides into the gene, a point mutation, e.g., a substitution of one or more nucleotides of the gene, a gross chromosomal rearrangement of the gene, e.g., a translocation, inversion, or deletion. For example, detecting the genetic lesion can include: (i) providing a probe/primer including an oligonucleotide containing a region of nucleotide sequence which hybridizes to a sense or antisense sequence from the gene or naturally occurring mutants thereof or 5′ or 3′ flanking sequences naturally associated with the gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA transcript of the gene; the presence of a non-wild type splicing pattern of a messenger RNA transcript of the gene; or a non-wild type level of the gene.

Methods of the invention can be used prenatally or to determine if a subject's offspring will be at risk for a disorder.

In preferred embodiments the method includes determining the structure of a gene involved in VEGF-KDR signaling (e.g., VEGF, KDR, PI3 kinase, PKC-zeta), an abnormal structure being indicative of risk for the disorder.

In preferred embodiments the method includes contacting a sample form the subject with an antibody to the protein or a nucleic acid, which hybridizes specifically with the gene. These and other embodiments are discussed below.

The presence, level, or absence of protein or nucleic acid identified by a method described herein in a biological sample can be evaluated by obtaining a biological sample from a test subject and contacting the biological sample with a compound or an agent capable of detecting the protein or nucleic acid (e.g., mRNA, genomic DNA) that encodes the protein such that the presence of protein or nucleic acid is detected in the biological sample. The term “biological sample” includes tissues, cells and biological fluids isolated from a subject, as well as tissues, cells and fluids present within a subject. A preferred biological sample is a retinal cell or tissue, e.g., a retinal endothelial or pericyte cell or tissue. The level of expression of the gene can be measured in a number of ways, including, but not limited to: measuring the mRNA encoded by the gene; measuring the amount of protein encoded by the gene; or measuring the activity of the protein encoded by the gene.

The level of mRNA corresponding to the gene in a cell can be determined both by in situ and by in vitro formats.

In another aspect, the invention features diagnostic methods to determine if a subject is at risk for a hypertension related disorder, e.g., a retinal disorder, e.g., retinopathy. The methods include one or more of the detection steps described above. In a preferred embodiment, the subject has or is at risk for diabetes, e.g., Type I or Type II diabetes, or other disorders which when combined with hypertension can result in hypertension related disorders such as diabetic retinopathy.

In another aspect, the invention features a method of modulating cell proliferation induced by hypertension. The method includes administering an effective amount of an agent described herein that reduces VEGF-KDR signaling, e.g., an agent described herein that reduces VEGF, KDR, PI3 kinase and/or PKC-zeta expression, levels,and/or activity.

In one embodiment, the agent reduces VEGF-KDR signaling in a cell subjected to hypertension. The cell can be an endothelial cell, e.g., a cardiac endothelial cell, a retinal endothelial cell or a pericyte. In another embodiment, the agent reduces VEGF-KDR signaling in a cell subjected to a mechanical stress, e.g., stretch. In one embodiment, the cell is subject to mechanical stress as a result of an increase in vascular pressure, e.g., increased blood pressure or hypertension. The cell can be in a live organism, e.g., an experimental model, or a patient. The experimental animal can be a rat, e.g., a spontaneous hypertensive rat. In another embodiment, the cell is subjected to mechanical stress as result of mechanical strain from a device, e.g., a vacuum stretch apparatus. In a preferred embodiment, the strain is produce with a cardiac profile, e.g., with a frequency of about 60 cpm. The strain can uniform in radial and circumferential dimensions. In one embodiment, the cell is a bovine retinal microvascular endothelial cell (BREC).

An agent which reduces VEGF-KDR signaling can be an agent which modulates a VEGF-KDR signaling component. In one embodiment, the signaling component is VEGF. In another embodiment, the signaling component is a VEGF receptor, e.g., KDR. In another embodiment, the signaling component is PKC-zeta. In yet another embodiment, the signaling component is PI3 kinase. Agents that decrease the expression, levels or activity of such VEGF-KDR signaling components are described, e.g., herein above.

These invention provide methods of reducing symptoms of hypertension by inhibiting VEGF expression and/or activity, KDR expression and/or activity, and PKC-zeta expression and/or activity. In particular, the invention can be used to treat disorders and diseases which are exacerbated by hypertension, such disorders and diseases can include retinal vascular disorders, such as, diabetic retinopathy. The invention can be used to reduce hypertension-exacerbated effects on tissues such as that observed with hypertensive retinopathy.

A “test compound” can be any chemical compound, for example, a small organic molecule, a carbohydrate, a lipid, an amino acid, a polypeptide, a nucleoside, a nucleic acid, or a peptide nucleic acid. The test compound or compounds can be naturally occurring, synthetic, or both. A test compound can be the only substance assayed by the method described herein. The test compound can be a single compound, or a member of a collection of compounds, e.g., a member of a combinatorial library. For example, a collection of test compounds can be assayed either consecutively or concurrently by the methods described herein. In a preferred embodiment, a high-throughput screen is used to screen test compounds.

“Treatment” or “treating a subject” is defined as the application or administration of a therapeutic agent to a patient, or application or administration of a therapeutic agent to an isolated tissue or cell line from a patient who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small molecules, proteins, peptides, antibodies, ribozymes and nucleci acids, e.g., antisense oligonucleotides.

Other embodiments are within the following description and the claims.

A “heterologous promoter”, as used herein is a promoter which is not naturally associated with a gene or a purified nucleic acid.

A “purified” or “substantially pure” or isolated “preparation” of a polypeptide, as used herein, means a polypeptide that has been separated from other proteins, lipids, and nucleic acids with which it naturally occurs. Preferably, the polypeptide is also separated from substances, e.g., antibodies or gel matrix, e.g., polyacrylamide, which are used to purify it. Preferably, the polypeptide constitutes at least 10, 20, 50 70, 80 or 95% dry weight of the purified preparation. Preferably, the preparation contains: sufficient polypeptide to allow protein sequencing; at least 1, 10, or 100 μg of the polypeptide; at least 1, 10, or 100 mg of the polypeptide.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

The terms “peptides”, “proteins”, and “polypeptides” are used interchangeably herein.

The term “small molecule”, as used herein, includes peptides, peptidomimetics, or nonpeptidic compounds, such as organic molecules, having a molecular weight less than 2000, preferably less than 1000. Methods described herein can be used to screen small molecules.

As used herein, the term “transgene” means a nucleic acid sequence (encoding, e.g., one or more subject PKC isoform), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of the selected nucleic acid, all operably linked to the selected nucleic acid, and may include an enhancer sequence.

As used herein, the term “transgenic cell” refers to a cell containing a transgene.

As used herein, a “transgenic animal” is any animal in which one or more, and preferably essentially all, of the cells of the animal includes a transgene. The transgene can be introduced into the cell, directly or indirectly by introduction into a precursor of the cell, by way of deliberate genetic manipulation, such as by microinjection or by infection with a recombinant virus. This molecule may be integrated within a chromosome, or it may be extra chromosomally replicating DNA.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue, such as vascular or heart tissue. The term also covers so-called “leaky” promoters, which regulate expression of a selected DNA primarily in one tissue, but cause expression in other tissues as well.

“Misexpression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

### DETAILED DESCRIPTION

The data described herein demonstrate that cyclic stretch and hypertension induce expression, e.g., retinal expression, of VEGF and KDR. Because stretch-induced VEGF-KDR signaling is dependent on PI3 kinase and PKC-zeta, treatments that target VEGF, KDR, PI3 kinase and/or PKC-zeta expression, levels or activity can prove therapeutically effective for hypertension and related disorders and conditions, such as hypertensive retinopathy or diabetic retinopathy.

Cyclic Stretch and hypertension induce retinal expression of VEGF and VEGF-receptor 2 (KDR

Because systemic hypertension increases vascular stretch, we evaluated the expression of VEGF, VEGF-R2 (KDR), and VEGF-R1 (fins-like tyrosine kinase [Flt]) in bovine retinal endothelial cells (BRECs) undergoing clinically relevant cyclic stretch and in spontaneously hypertensive rat (SHR) retina. See Examples 1-7 herein below.

A single exposure to 20% symmetric static stretch increased KDR mRNA expression 3.9 +/−1.1-fold after 3 h (P=0.002), with a gradual return to baseline within 9 h. In contrast, BRECs exposed to cardiac-profile cyclic stretch at 60 cpm continuously accumulated KDR mRNA in a transcriptionally mediated, time-dependent and stretch-magnitude-dependent manner. Exposure to 9% cyclic stretch increased KDR mRNA expression 8.7+/−2.9-fold (P=0.011) after 9 h and KDR protein concentration 1.8+/−0.3-fold (P=0.005) after 12 h. Stretched-induced VEGF responses were similar. Scatchard binding analysis demonstrated a 180+/−40% (P=0.032) increase in high-affinity VEGF receptor number with no change in affinity. Cyclic stretch increased basal thymidine uptake 60+/−10% (P<0.001) and VEGF-stimulated thymidine uptake by 2.6+/−0.2-fold (P=0.005). VEGF-NAb reduced cyclic stretch-induced thymidine uptake by 65%. Stretched-induced KDR expression was not inhibited by AT1 receptor blockade using candesartan. Hypertension increased retinal KDR expression 67 +/−42% (P<0.05) in SHR rats compared with normotensive WKY control animals. When hypertension was reduced using captopril or candesartan, retinal KDR expression returned to baseline levels. VEGF reacted similarly, but Flt expression did not change. These data (See Examples suggest a novel molecular mechanism that accounts for the exacerbation of diabetic retinopathy by concomitant hypertension, and may partially explain the principal clinical manifestations of hypertensive retinopathy itself.

Stretch-Induced Retinal VEGF Expression is Mediated by PI3 Kinase and PKC-zeta

The time course of VEGF expression in response to static and cyclic stretch in retinal pericytes was similar to that observed in retinal endothelial cells, although the magnitude of the response was approximately one third of that in endothelial cells. Cyclic stretch induced rapid increases in ERK 1/2 phosphorylation, PI3 kinase activity, Akt phosphorylation and PKC-zeta activity. However, the ERK 1/2 -independence of stretch-induced VEGF expression was substantiated by several findings. Stretch-induced VEGF mRNA expression was not suppressed by either PD98059 or adenovirus infection with dominant negative ERK. Overexpression of wild type ERK did not increase basal or stretch-induced VEGF expression. Furthermore, stretch-induced ERK1/2 activation was mediated by classical/novel isoforms of PKC and Ras (as evidenced by inhibition of the response by classical/novel PKC isoforms inhibitor GF109203X and overexpression of dominant negative Ras) but not mediated by PI3 kinase, tyrosine kinases or PKC-zeta (as evidenced by lack of response to wortmannin & LY294002, genistein, or overexpression of wild type and dominant negative PKC-zeta, respectively). In contrast, the opposite results were obtained when evaluating these interventions on stretch-induced VEGF expression. These data demonstrate that although stretch activates several signaling pathways, VEGF expression is mediated by PI3 kinase and PKC-zeta in a ERK-, Ras- and classical/novel PKC isoform-independent manner. See Examples 8-11. In addition, direct modulation of ERK may not be adequate in itself to alter VEGF expression in these cells as evidenced by the lack of effect of ERK 1/2 inhibitors and wild type or dominant negative ERK expression. It should be noted; however, that overexpression of wild type ERK 1/2 might not have a major impact on the basal state if it is not significantly activated.

ERK has been reported as important in VEGF expression induced by starvation in human colon carcinoma cells; v-ras, v-raf and c-myc transformation of rat liver epithelial cells; PMA treatment in human glioblastoma U373 cells; Ras expression in human fibrosarcoma and renal cell carcinoma cell lines; endothelin stimulation of human vascular smooth muscle cells; and von Hippel-Lindau tumor suppressor gene action. Hypoxic induction of VEGF may also involve ERK since inhibition of Raf-1 markedly reduces VEGF induction. However, hypoxia can be additive to VEGF expression induced by ERK 1/2 activation in hamster fibroblasts where a single inhibitor of ERK did not suppress hypoxia-induced VEGF expression. The ERK independence observed in the system described herein suggests that VEGF expression in response to different stimuli may be mediated by a variety of signaling pathways and/or may reflect a potential uniqueness of retinal pericytes.

The importance of the atypical PKC-zeta isoform in mediating stretch-induced VEGF expression is underscored by several findings described herein. PKC-zeta protein expression was present in retinal endothelial cells and present in even higher amounts in retinal pericytes. PKC-zeta activity was increased nearly 3-fold by cyclic stretch. Stretch-induced VEGF expression was inhibited by expression of dominant negative PKC-zeta and increased by overexpression of wild type PKC-zeta. In contrast, overexpression of wild type classical PKC-α isoform or novel PKC-δ isoform did not effect VEGF expression. The activation of PKC-zeta within 15 minutes of stretch onset is consistent with previous time course data for PKC-zeta activation following exposure to insulin (10-20 min), NGF (9-15 min), or hypoxia-reperfusion (15 min).

In other systems, including insulin-stimulated rat adipocytes, reoxygenation of rat cardiomyocytes and endotoxin-treated human alveolar macrophages (Sajan et al. (1999) J. Biol. Chem. 274:30495-30500; Mizukami et al. (2000) J. Biol. Chem. 275:19921-19927; Monick et al. (2000) J. Immunol. 165, 4632-4639), PI3 kinase activation induces ERK activity through a PKC-zeta mediated pathway. However, the data described herein suggest that stretch-induced activation of ERK 1/2 in retinal pericytes is mediated by a different mechanism since inhibition of PKC-zeta using dominant negative adenovirus did not prevent stretch-induced ERK 1/2 phosphorylation.

Although these are the first studies to elucidate the role of PKC-zeta in stretch-induced VEGF expression, PKC-zeta has been previously implicated as a modulator of VEGF (Pal et al. (1998) J. Biol. Chem. 273, 26277-26280; Pal et al. (1997) J. Biol. Chem. 272:27509-27512) Overexpression of PKC-zeta in human glioblastoma U373 cells increased VEGF mRNA expression (Shih et al. (1999) J. Biol. Chem. 274:15407-15414). The von Hippel-Lindau tumor suppressor gene has been shown to form cytoplasmic complexes with PKC-δ and PKC-zeta, preventing their translocation to the cell membrane and reducing the constitutive overexpression of VEGF characteristically observed in sporadic renal cell carcinomas (RCC). In addition, PKC-zeta binds and phosphorylates transcription factor SP1 in RCC, resulting in VEGF expression. Ras-induced VEGF expression in human fibrosarcoma and renal cell carcinoma cell lines is almost totally dependent on PKC-zeta activity (Pal et al. (2110). J. Biol. Chem. 276:2395-2403). However, as discussed above, ERK was an important component of these pathways.

The role of PI3 kinase in stretch-induced VEGF expression and Akt phosphorylation is supported in the data described herein by the inhibitory effect of two different PI3 kinase inhibitors (wortmannin and LY294002) and dominant negative expression of the p85 subunit of PI3 kinase. In addition, wortmannin completely inhibited stretch-induced PKC-zeta activity. However, Akt did not appear to mediate stretch-induced VEGF expression, as expression of dominant negative or constitutively active Akt had no effect. This finding differs from that observed in chicken cells where overexpression of myristylated Akt increased basal VEGF expression and restored VEGF expression in cells after PI3 kinase inhibition (Jiang et al. (2000). Proc Natl. Acad. Sci. USA 97:1749-1753). Thus, the role of Akt in mediating VEGF expression may be cell type and/or stimuli-dependent. The studies described herein do not eliminate the possibility that stretch-induced Akt may be involved in late stages of VEGF expression but do suggest that at least for stretch-induced VEGF expression, the PKC-zeta pathway, independent of Akt activation, predominates within in first several hours in, e.g., retinal pericytes.

Stretch can induce the expression of numerous genes through activation of various intracellular pathways including membrane K+ channels, G proteins, intracellular Ca2+, cAMP, cGMP, inositol triphosphate, protein kinase C, MAP kinase, protein tyrosine kinases, focal adhesion kinase, and alterations in intracellular redox state (Lehoux et al. (1998) Hypertension 32, 338-345 (1998); Li et al. (1999) J. Biol. Chem. 274, 25273-25280; Hishikawa et al. (1997) Circ. Res. 81, 797-803). Fluid shear stress can also mediate signaling through activation of heterotrimeric and small G-proteins, resulting in ERK 1/2 and phospholipase C activation, with subsequent IP3 and DAG generation, Ca2+ release and PKC activation. However, this mechanism may not be involved in stretch-induced VEGF expression due to the noted ERK 1/2 independence and involvement of PKC-zeta, a Ca2+ independent isoform of PKC. Interestingly, mechanical stretch can directly induce growth factor receptor autophosphorylation presumably through changes in cellular morphology leading to altered receptor conformation and subsequent exposure of the kinase domain (Hu et al. (1998). FASEB J. 12, 1135-1142). PDGF receptor can be activated by stretch independently of its ligand. Our data demonstrating stretch increases PDGFR-B tyrosine phosphorylation and subsequent p85 association suggests that such as response may mediate stretch-induced activation of PI3 kinase.

Since mechanical stretch can regulate gene expression in a variety of ways and since hypertension increases retinal arterial diameter up to 35%, hypertension-induced stretch in vivo may increase VEGF expression enough to exacerbate ocular conditions characterized by endothelial proliferation and leakage such as diabetic retinopathy. Indeed, as described herein, retinal expression of VEGF and VEGF-R2 are increased in spontaneously hypertensive rats. Although the magnitude of stretch experienced by the vasculature is likely to diminish as the internal capillary diameter becomes smaller, our studies did not identify a maximal VEGF mRNA accumulation as expression continued to increase after all durations of cardiac profile cyclic stretch. Thus, it is possible that even very small increases in cyclic stretch could eventually result in significantly increased VEGF expression.

This finding may also be important, as retinal pericytes are characteristically lost early in the course of diabetic retinopathy. Thus, even with diminishing numbers, significant localized VEGF expression may be present. Retinal pericytes are an important cell type especially in early stages of retinopathy as they regulate retinal vascular tone and perfusion, mediate diabetes-induced alterations in autoregulation of retinal blood flow and vasoreactivity and produce VEGF. In addition, retinal endothelial cells, which are not compromised until later stages of diabetic retinopathy, respond to stretch with very similar expression of VEGF as do pericytes.

In summary, we demonstrate herein that cardiac profile cyclic stretch induces VEGF expression via PI3 kinase-mediated activation of PKC-zeta. Furthermore, stretch-induced VEGF expression is independent of ERK 1/2, Ras, classical/novel isoforms of PKC and Akt despite stretch-induced activation of these molecules. In addition, PKC-zeta activation does not mediate ERK 1/2 activation. Since each these molecules have been implicated as mediators of VEGF expression in response to other perturbations, these data suggest that a variety of pathways may be involved in mediating increased VEGF expression in response to diverse stimuli in various cell types. Furthermore, these studies identify new therapeutic targets with potential to ameliorate the well-documented clinical exacerbation of ocular diseases, such as diabetic retinopathy, by concomitant hypertension.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning-A Laboratory Manual, 3d Ed., ed. by Sambrook et al. (Cold Spring Harbor Laboratory Press: 2001); DNA Cloning, Volumes I and II (D. N. Glover ed., 1985); Oligonucleotide Synthesis (M. J. Gait ed., 1984); Mullis et al. U.S. Pat. No.: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

The invention also provides methods for identifying modulators, i.e., candidate or test compounds or agents (e.g., proteins, peptides, peptidomimetics, peptoids, small molecules or other drugs) which have an inhibitory effect on, for example, the expression or activity of a component of the VEGF-KDR signaling pathway, e.g., VEGF, KDR, PI3 kinase, PKC-zeta, thereby decreasing hypertension or a related disorder. Compounds thus identified can be used to treat hypertension or a related disorder in a method described herein.

Generation of Analogs: Production of Altered DNA and Peptide Sequences by Random Methods

Amino acid sequence variants of a protein, e.g., a VEGF, KDR, PI3 kinase and/or PKC-zeta agonist or antagonist, can be prepared by random mutagenesis of DNA which encodes a protein or a particular domain or region of a protein. Useful methods include PCR mutagenesis and saturation mutagenesis. A library of random amino acid sequence variants can also be generated by the synthesis of a set of degenerate oligonucleotide sequences. (Methods for screening proteins in a library of variants, e.g., screening for a VEGF, KDR, PI3 kinase and/or PKC-zeta modulating activity, are elsewhere herein.)

PCR Mutagenesis

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, SA (1983) Tetrahedron 39:3; Itakura et al. (1981) Recombinant DNA, Proc 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Generation of Analogs: Production of Altered DNA and Peptide Sequences by Directed Mutagenesis

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. (1978) USA, 75: 5765).

Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate mutants. For example, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Primary High-Through-Put Methods for Screening Libraries of Peptide Fragments or Homologs

Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, assembly into a trimeric molecules, binding to natural ligands, e.g., a receptor or substrates, facilitates relatively easy isolation of the vector encoding the gene whose product was detected. Each of the techniques described below is amenable to high through-put analysis for screening large numbers of sequences created, e.g., by random mutagenesis techniques.

Two Hybrid Systems

Two hybrid (interaction trap) assays can be used to identify a protein that interacts with a component of the VEGF-KDR signaling pathway, e.g., VEGF, KDR, PI3 kinase, PKC-zeta. These may include agonists, superagonists, and antagonists of a component of the VEGF-KDR signaling pathway, e.g., VEGF, KDR, PI3 kinase, PKC-zeta. (The subject protein and a protein it interacts with are used as the bait protein and fish proteins.). These assays rely on detecting the reconstitution of a functional transcriptional activator mediated by protein-protein interactions with a bait protein. In particular, these assays make use of chimeric genes which express hybrid proteins. The first hybrid comprises a DNA-binding domain fused to the bait protein. e.g., a VEGF, KDR, PI3 kinase, or PKC-zeta molecule or a fragment thereof. The second hybrid protein contains a transcriptional activation domain fused to a “fish” protein, e.g. an expression library. If the fish and bait proteins are able to interact, they bring into close proximity the DNA-binding and transcriptional activator domains. This proximity is sufficient to cause transcription of a reporter gene which is operably linked to a transcriptional regulatory site which is recognized by the DNA binding domain, and expression of the marker gene can be detected and used to score for the interaction of the bait protein with another protein.

Display Libraries

In one approach to screening assays, the candidate peptides are displayed on the surface of a cell or viral particle, and the ability of particular cells or viral particles to bind an appropriate receptor protein via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, a detectably labeled ligand can be used to score for potentially functional peptide homologs. Fluorescently labeled ligands, e.g., receptors, can be used to detect homolog which retain ligand-binding activity. The use of fluorescently labeled ligands, allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and f1 are most often used in phage display libraries. Either of the phage gIII or gVIII coat proteins can be used to generate fusion proteins without disrupting the ultimate packaging of the viral particle. Foreign epitopes can be expressed at the NH₂-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J. Biol. Chem. 267:16007-16010; Griffiths et al. (1993) EMBO J 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus-a conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al. (1988) Bio/Tech. 6, 1080-1083). Surface proteins of other bacterial species have also served as peptide fusion partners. Examples include the Staphylococcus protein A and the outer membrane protease IgA of Neisseria (Hansson et al. (1992) J Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. U.S.A. 89-1869)

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J Med. Chem. 37(9):1233-1251). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

Secondary Screens

The high through-put assays described above can be followed by secondary screens in order to identify further biological activities which will, e.g., allow one skilled in the art to differentiate agonists from antagonists. The type of a secondary screen used will depend on the desired activity that needs to be tested. For example, an assay can be developed in which the ability to inhibit an interaction between a protein of interest (e.g., a component of the VEGF-KDR signaling pathway, e.g., VEGF, KDR, PI3 kinase, PKC-zeta) and a ligand (e.g., a PKC zeta substrate) can be used to identify antagonists from a group of peptide fragments isolated though one of the primary screens described above.

Therefore, methods for generating fragments and analogs and testing them for activity are known in the art. Once the core sequence of interest is identified, it is routine to perform for one skilled in the art to obtain analogs and fragments.

Peptide Mimetics

The invention also provides for reduction of the protein binding domains of the subject polypeptides, e.g., a component of the VEGF-KDR signaling pathway, e.g., VEGF, KDR, PI3 kinase, PKC-zeta, to generate mimetics, e.g. peptide or non-peptide agents. See, for example, “Peptide inhibitors of human papillomavirus protein binding to retinoblastoma gene protein” European patent applications EP 0 412 762 and EP 0 031 080.

Non-hydrolyzable peptide analogs of critical residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffinan et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

Antibodies

The invention also includes antibodies specifically reactive with a gene involved in VEGF-KDR signaling, e.g., VEGF, KDR, PI3 kinase, PKC-zeta described herein. An antibody can be an antibody or a fragment thereof, e.g., an antigen binding portion thereof. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E.A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The term “antigen-binding fragment” of an antibody (or simply “antibody portion,” or “fragment”), as used herein, refers to one or more fragments of a full-length antibody that retain the ability to specifically bind to an antigen (e.g., a polypeptide encoded by a nucleic acid of Group I or II). Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate nucleic acids, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

Anti-protein/anti-peptide antisera or monoclonal antibodies can be made as described herein by using standard protocols (See, for example, Antibodies: A Laboratory Manual ed. by Harlow and Lane (Cold Spring Harbor Press: 1988)).

Molecules involved in VEGF-KDR signaling (e.g., VEGF, KDR, PI3 kinase, PKC-zeta) can be used as an immunogen to generate antibodies that bind the component using standard techniques for polyclonal and monoclonal antibody preparation. The full-length component protein can be used or, alternatively, antigenic peptide fragments of the component can be used as immunogens.

Typically, a peptide is used to prepare antibodies by immunizing a suitable subject, (e.g., rabbit, goat, mouse or other mammal) with the immunogen. An appropriate immunogenic preparation can contain, for example, a recombinant VEGF-KDR signaling molecule, e.g., VEGF, KDR, PI3 kinase, or PKC-zeta, e.g., a VEGF, KDR, PI3 kinase, or PKC-zeta, peptide, or a chemically synthesized VEGF, KDR, PI3 kinase, or PKC-zeta signaling molecule, e.g., a VEGF, KDR, PI3 kinase, or PKC-zeta peptide or anagonist. See, e.g., U.S. Pat. No. 5,460,959; and co-pending U.S. applications U.S. Ser. No. 08/334,797; U.S. Ser. No. 08/231,439; U.S. Ser. No. 08/334,455; and U.S. Ser. No. 08/928,881 which are hereby expressly incorporated by reference in their entirety. The nucleotide and amino acid sequences of the VEGF-KDR signaling molecules described herein are known. The preparation can further include an adjuvant, such as Freund's complete or incomplete adjuvant, or similar immunostimulatory agent. Immunization of a suitable subject with an immunogenic VEGF-KDR signaling molecule preparation induces a polyclonal anti- VEGF-KDR signaling molecule antibody response.

Additionally, antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., Science 240:1041-1043, 1988; Liu et al., PNAS 84:3439-3443, 1987; Liu et al., J. Immunol. 139:3521-3526, 1987; Sun et al. PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988); Morrison, S. L., Science 229:1202-1207, 1985; Oi et al., BioTechniques 4:214, 1986; Winter U.S. Pat. No.5,225,539; Jones et al., Nature 321:552-525, 1986; Verhoeyan et al., Science 239:1534, 1988; and Beidler et al., J. Immunol. 141:4053-4060, 1988.

In addition, a human monoclonal antibody directed against a VEGF-KDR signaling molecule, e.g., a VEGF, KDR, PI3 kinase, or PKC-zeta described herein, can be made using standard techniques. For example, human monoclonal antibodies can be generated in transgenic mice or in immune deficient mice engrafted with antibody-producing human cells. Methods of generating such mice are describe, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. Pct publication WO 94/04667; Ditullio et al. PCT publication WO 95/17085; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Choi et al. (1993) Nature Genet. 4:117-123; Tuaillon et al. (1993) PNAS 90:3720-3724; Bruggeman et al. (1991) Eur J Immunol 21:1323-1326); Duchosal et al. PCT publication WO 93/05796; U.S. Pat. No. 5,411,749; McCune et al. (1988) Science 241:1632-1639), Kamel-Reid et al. (1988) Science 242:1706; Spanopoulou (1994) Genes & Development 8:1030-1042; Shinkai et al. (1992) Cell 68:855-868). A human antibody-transgenic mouse or an immune deficient mouse engrafted with human antibody-producing cells or tissue can be immunized with a VEGF-KDR signaling molecule, e.g., a VEGF, KDR, PI3 kinase, or PKC-zeta described herein or an antigenic peptide thereof, and splenocytes from these immunized mice can then be used to create hybridomas. Methods of hybridoma production are well known.

Human monoclonal antibodies against a VEGF-KDR signaling molecule, e.g., a VEGF, KDR, PI3 kinase, or PKC-zeta described herein, can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581-597; and Griffths et al. (1993) EMBO J 12:725-734. In addition, a combinatorial library of antibody variable regions can be generated by mutating a known human antibody. For example, a variable region of a human antibody known to bind a VEGF-KDR signaling molecule can be mutated, by for example using randomly altered mutagenized oligonucleotides, to generate a library of mutated variable regions which can then be screened to bind to a VEGF—KDR signaling molecule, e.g., a VEGF, KDR, PI3 kinase, or PKC-zeta. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457-4461. The immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) supra; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. Once displayed on the surface of a display package (e.g., filamentous phage), the antibody library is screened to identify and isolate packages that express an antibody that binds a VEGF-KDR signaling molecule, e.g., a a VEGF, KDR, PI3 kinase, or PKC-zeta, described herein. In a preferred embodiment, the primary screening of the library involves panning with an immobilized VEGF-KDR signaling molecule, e.g., a a VEGF, KDR, PI3 kinase, or PKC-zeta described herein and display packages expressing antibodies that bind immobilized proteins described herein are selected.

Antisense Nucleic Acid Sequences

Nucleic acid molecules which are antisense to a nucleotide encoding a component of the VEGF-KDR signaling pathway, e.g., VEGF, KDR, PI3 kinase, PKC-zeta, can be used as an agent which reduces hypertension or a related disorder. An “antisense” nucleic acid includes a nucleotide sequence which is complementary to a “sense” nucleic acid encoding the component, e.g., complementary to the coding strand of a double-stranded cDNA molecule or complementary to an mRNA sequence. Accordingly, an antisense nucleic acid can form hydrogen bonds with a sense nucleic acid. The antisense nucleic acid can be complementary to an entire coding strand, or to only a portion thereof. For example, an antisense nucleic acid molecule which antisense to the “coding region” of the coding strand of a nucleotide sequence encoding the component can be used.

The coding strand sequences encoding PKC isozymes described herein are known. Given the coding strand sequences encoding these isozymes, antisense nucleic acids can be designed according to the rules of Watson and Crick base pairing. The antisense nucleic acid molecule can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′ methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2, 6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

Administration

An agent which modulates the level of expression of a a component of the VEGF-KDR signaling pathway, e.g., VEGF, KDR, PI3 kinase, PKC-zeta, described herein can be administered to a subject by standard methods. For example, the agent can be administered by any of a number of different routes including intravenous, intradermal, subcutaneous, oral (e.g., inhalation), transdermal (topical), and transmucosal. In one embodiment, the modulating agent can be administered orally. In another embodiment, the agent is administered by injection, e.g., intramuscularly, or intravenously.

The agent which modulates protein levels, e.g., nucleic acid molecules, polypeptides, fragments or analogs, modulators, and antibodies (also referred to herein as “active compounds”) can be incorporated into pharmaceutical compositions suitable for administration to a subject, e.g., a human. Such compositions typically include the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions. A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a PKC β polypeptide or anti-PKC β antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The nucleic acid molecules described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al.,i PNAS 91:3054-3057, 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

Gene Therapy

The nucleic acids described herein, or an antisense nucleic acid, can be incorporated into gene constructs to be used as a part of a gene therapy protocol to deliver nucleic acids encoding either an agonistic or antagonistic form of a protein described herein, e.g., a component of the VEGF-KDR signaling pathway, e.g., VEGF, KDR, PI3 kinase, PKC-zeta. The invention features expression vectors for in vivo transfection and expression of nucleic acids described herein in particular cell types so as to reconstitute the function of, or alternatively, antagonize the function of the component in a cell in which that polypeptide is misexpressed. Expression constructs of such components may be administered in any biologically effective carrier, e.g. any formulation or composition capable of effectively delivering the component gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO4 precipitation carried out in vivo.

A preferred approach for in vivo introduction of nucleic acid into a cell is by use of a viral vector containing nucleic acid, e.g. a cDNA, encoding a PKC described herein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirusderived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adenoassociated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol.158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). In addition to viral transfer methods, such as those illustrated above, non-viral methods can also be employed to cause expression of a PKC described herein in the tissue of a subject. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et. al. (2000) Gene Ther 7(21):1867-74.

In a representative embodiment, a gene encoding a a component of the VEGF-KDR signaling pathway, e.g., VEGF, KDR, PI3 kinase, PKC-zeta, can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Cell Therapy

A component of the VEGF-KDR signaling pathway, e.g., VEGF, KDR, PI3 kinase, PKC-zeta, described herein can also be increased in a subject by introducing into a cell, e.g., an endothelial cell, a nucleotide sequence that modulates the production of a component of the VEGF-KDR signaling pathway, e.g., VEGF, KDR, PI3 kinase, PKC-zeta, e.g., a nucleotide sequence encoding a polypeptide or functional fragment or analog thereof, a promoter sequence, e.g., a promoter sequence from a PKC zeta gene or from another gene; an enhancer sequence, e.g., 5′ untranslated region (UTR), e.g., a 5′ UTR from a PKC gene or from another gene, a 3′ UTR, e.g., a 3′ UTR from a PKC-zeta gene or from another gene; a polyadenylation site; an insulator sequence; or another sequence that modulates the expression of a component of the VEGF-KDR signaling pathway, e.g., VEGF, KDR, PI3 kinase, PKC-zeta. The cell can then be introduced into the subject.

Primary and secondary cells to be genetically engineered can be obtained form a variety of tissues and include cell types which can be maintained propagated in culture. For example, primary and secondary cells include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types. Primary cells are preferably obtained from the individual to whom the genetically engineered primary or secondary cells are administered. However, primary cells may be obtained for a donor (other than the recipient).

The term “primary cell” includes cells present in a suspension of cells isolated from a vertebrate tissue source (prior to their being plated i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of secondary cells which have been passaged one or more times.

Primary or secondary cells of vertebrate, particularly mammalian, origin can be transfected with an exogenous nucleic acid sequence which includes a nucleic acid sequence encoding a signal peptide, and/or a heterologous nucleic acid sequence, e.g., encoding a PKC described herein, e.g., PKC β , e.g., PKC β 1, or an agonist or antagonist thereof, and produce the encoded product stably and reproducibly in vitro and in vivo, over extended periods of time. A heterologous amino acid can also be a regulatory sequence, e.g., a promoter, which causes expression, e.g., inducible expression or upregulation, of an endogenous sequence. An exogenous nucleic acid sequence can be introduced into a primary or secondary cell by homologous recombination as described, for example, in U.S. Pat. No.: 5,641,670, the contents of which are incorporated herein by reference. The transfected primary or secondary cells may also include DNA encoding a selectable marker which confers a selectable phenotype upon them, facilitating their identification and isolation.

Vertebrate tissue can be obtained by standard methods such a punch biopsy or other surgical methods of obtaining a tissue source of the primary cell type of interest. For example, punch biopsy is used to obtain skin as a source of fibroblasts or keratinocytes. A mixture of primary cells is obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAEdextrin-mediated transfection, lipofection or electrophoration, all of which are routine in the art. Transfected primary or secondary cells undergo sufficient number doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. One implanted in individual, the transfected cells produce the product encoded by the heterologous DNA or are affected by the heterologous DNA itself. For example, an individual who suffers from an insulin related disorder is a candidate for implantation of cells producing an antagonist of PKC β described herein.

An immunosuppressive agent e.g., drug, or antibody, can be administered to a subject at a dosage sufficient to achieve the desired therapeutic effect (e.g., inhibition of rejection of the cells). Dosage ranges for immunosuppressive drugs are known in the art. See, e.g., Freed et al. (1992) N. Engl. J. Med. 327:1549; Spencer et al. (1992) N. Engl. J. Med. 327:1541’ Widner et al. (1992) n. Engl. J. Med. 327:1556). Dosage values may vary according to factors such as the disease state, age, sex, and weight of the individual.

### EXAMPLES

### Example 1

### Stretch Induces KDR mRNA and protein in BREC cells.

BRECs exposed to 20% static stretch increased KDR mRNA expression 3.9±1.1 fold; levels returned to baseline within 9 hrs. BRECS treated with 3%, 6%, and 9% uniform radial and circumferential strain all had concomitant elevation in KDR mRNA levels. Notably, BREC exposed to 9% cardiac cyclic stretch at 60 cycles/min continuously increased KDR mRNA expression (9.7±2.9 fold at 9 hours, p=0.011) in a time and magnitude dependent manner.. The increase in KDR mRNA expression was a result of transcriptional induction, not a change in MRNA stability (i.e., cyclic stretch-induced KDR mRNA expression is primarily transcriptionally mediated).

Cyclic stretch (9%/60 cpm) increased KDR protein concentration 1.8±0.3 fold (p=0.05) after 12 hrs as detected by antibodies against KDR. Scatchard binding analysis demonstrated a 181±40% increase (p=0.03) in VEGF binding sites on the cell surface. This results indicate that the number of KDR protein molecules on the cell surface is increased 1.8 fold rather than there being an alteration in affinity of the molecules for VEGF.

### Example 2

### Stretch Induced VEGF mRNA

BRECS treated with 6% uniform radial and circumferential strain demonstrated a greater than 8 fold increase in VEGF mRNA expression. Thus stretching increases both the levels of VEGF, a potent regulator of the vasculature, but also KDR, one of the VEGF receptors.

### Example 3

### Hypertension Induces KDR expression in Retina of Animals

To determine the effect of hypertension on KDR levels in vivo, KDR mRNA was quantified in hypertensive rats (SHR) as well as weight-matched controls (WKY). KDR mRNA levels were elevated in the retinal cells of hypertensive rats relative to normal controls. Another VEGF receptor, Flt-1, however, was not affected by hypertension. Moreover, the elevation in KDR mRNA levels was blocked by treatment with captopril and by candesartan cilexetil. Captopril is an angiotensin converting enzyme (ACE) inhibitor. Candesartan Cilexetil is an angiotensin II antagonist. Thus, normalization of blood pressure using ACE (captopril) and AT1 receptor (candesartan cilexetil) inhibitors prevents the increase in retinal KDR expression.

### Example 4

### Hypertension Induces VEGF expression in Retina of Animals.

To determine the effect of hypertension on VEGF levels in vivo, VEGF mRNA was quantified in hypertensive rats as well as weight-matched controls. VEGF mRNA levels were elevated in the retinal cells of hypertensive rats relative to normal controls. Both KDR and VEGF mRNA are increased in hypertensive rat retina compared to non-hypertensive control animals. The elevation in VEGF mRNA levels was blocked by treatment with captopril, an ACE inhibitor, and by candesartan cilexetil, an angiotensin II antagonist. Thus, normalization of blood pressure using ACE(captopril) and AT1 receptor (candesartan cilexetil) inhibitors prevents the increase in retinal VEGF expression.

### Example 5

### Cyclic Stretch Increases Mitogenesis

Cells can respond to VEGF by proliferation. A measure of proliferation is the rate of cell progression through mitosis. Cells transiting S phase will incorporate ³H-thymidine. To determine whether a consequence of the increase in VEGF levels in response to cyclic stretching is mitogenesis, stretched and control cells were monitored for ³H-thymidine uptake. Cyclic stretching increased basal ³H-thymidine uptake 160±10% (p<0.001).

Thus, cyclic stretching in hypertension can result in cell proliferation, i.e. cyclic stretching increases the BREC growth response to VEGF. Cyclic stretch-induced BREC growth is predominantly mediated by VEGF.

### Example 6

### Induction of Mitogenesis by Cyclic Stretch is VEGF dependent

To determine if the effects of cyclic stretching on mitogenesis are mediated by VEGF signaling, cells were stretched while treated with a VEGF neutralizing antibody. The VEGF neutralizing antibody reduced cyclic stretch-induced mitogenesis by 65±10% (p=0.05). Moreover, cyclic stretching in combination to exogenous VEGF addition resulted in a 257±21% (p=0.005) increase in ³H-thymidine uptake; whereas addition of exogenous VEGF alone had no significant effect.

These findings indicate that cyclic stretching triggers mitogenesis by increased VEGF signaling. Elevated VEGF levels alone are insufficient; the additional responses to cyclic stretching, e.g., KDR induction, are required.

Cardiac profile cyclic stretch, at magnitudes readily observed clinically with hypertension, effectively increases KDR mRNA and protein expression in BRECs resulting in increased numbers of bioactive receptors that mediate stretch-and VEGF-induced mitogenesis. This response could account for the deleterious effects of hypertension of concomitant hypertension on diabetic retinopathy and other ocular disorders.

### Example 7

### Pathway of KDR mRNA Induction.

Cyclic stretch induces KDR mRNA at least 2.2 fold. This response to cyclic stretch is not significantly affected by candesartan cilexetil treatment. In contrast, angiotensin II which also induces KDR mRNA is blocked from doing so by candesartan cilexetil, an AT1 receptor antagonist. Thus, unlike Angiotensin II induced KDR expression, stretch induced KDR expression is not mediated through the AT1 receptor pathway.

### Example 8

### Characterization of stretch-induced VEGF expression in retinal capillary pericytes.

Confluent cultures of bovine retinal pericytes (BRPC) where subjected to a single instance of 5% or 20% static stretch for 0, 1, 3, 6, or 9 hours. Static stretch (20%) maximally increased VEGF mRNA expression 2.2-fold after 3 hours (p=0.048). VEGF mRNA levels gradually decline thereafter returning to baseline values after 6 hours. VEGF MRNA expression was increased 15±22%, 116±50% (p=0.048), 90±62% and −4±23% after 1, 3, 6 and 9 hours, respectively. VEGF mRNA expression in response to 5% static stretch was less pronounced with a tendency to increase within the first 3 hours; however, this change was not statistically significant.

The vasculature in vivo is continually exposed to repetitive stretch with pressure dynamics reflecting the cardiac cycle. To approximate this physiologically relevant condition, it was evaluated whether cardiac profile cyclic stretch altered VEGF mRNA expression in BRPC undergoing 9% and 3% cyclic stretch at a rate of 60 cpm with a dynamic stress contour reflecting that of the normal cardiac cycle. Cardiac cycle cyclic stretch increased VEGF mRNA expression in a time and dose-dependant manner. At 9% cyclic stretch, an increase in KDR mRNA expression was initially evident after 1 hour which continued to increase even after 9 hours when expression was 3.1±0.2 fold greater than in control cells (p<0.001). VEGF mRNA expression was increased 37±15%, 136±25% (p<0.001), 168±10% (p<0.001) and 206±17% (p<0.001) after 1, 3, 6 and 9 hours of cyclic stretch, respectively. Cyclic stretch of 3% also increased VEGF mRNA expression, although to a reduced extent with only a 1.7±0.6 fold increase observed after nine hours.

To determine if stretch-induced VEGF mRNA expression resulted in increased VEGF protein levels, cells were exposed to 9% stretch at 60 cpm for 12 hours. Cell lysates where evaluated by Western blot analysis. VEGF protein expression was increased 2.7±1.0 fold (p=0.002) as compared to control cells. Since stretch-induced mRNA expression could be the result of alterations in gene transcription or mRNA stability, BRPC were exposed to 9%/60 cpm cyclic stretch for 4 hours and then treated with 5 mg/ml Actinomycin D (5 μg/ml) and RNA was harvested 2 and 4 hours later. VEGF mRNA concentration declined at an equivalent rate in both control and stretched cells, suggesting that transcriptional regulation rather than changes in mRNA stability were primarily responsible for the stretch response.

### Example 9

### Evaluation of stretch-induced signaling pathways

To determine what pathways were activated in retinal pericytes exposed to cardiac profile cyclic stretch, ERK phosphorylation, PI3 kinase activity and Akt phosphorylation were evaluated. Stretch induced a rapid increase in ERK 1/ 2 phosphorylation which was initially evident after 2 minutes, maximal at 5 min (ERK1=20-fold; ERK2=8.9-fold increase) and still maintained above baseline even after 60 min (ERK1=6.3-fold; ERK2=4.5-fold). Both static and cyclic stretch resulted in similar ERK1/2 phosphorylation profiles. An excess of VEGFneutralizing antibody had no effect on stretch-induced ERK phosphorylation, suggesting that VEGF does not mediate this initial effect.

Cyclic stretch increased PI3 kinase activity by 2.6+0.8-fold at 5 min (p<0.05) and 1.8+0.4-fold after 15 min. Cyclic stretch also rapidly increased Akt phosphorylation, initially evident within 2 min (52+38%, p<0.05), reaching a maximum after 15 min (2.9+0.9-fold, p<0.01) and still evident after 60 min (2.05+0.6-fold, p<0.05). A potential mechanism underlying stretch-induced activation of PI3 kinase could be the effect of stretch on PDGF receptor B (PDGFR-B). Immunoprecipitation with antibody specific for PDGFR-B and subsequent immunoblotting with antibodies specific for phosphotyrosine or the p85 subunit of PI3 kinase showed stretch-induced phosphorylation of PDGFR-B and increased association with p85. Conversely, immunoprecipitation with phosphotyrosine specific antibody and subsequent immunoblotting with antibodies specific for PDGFR-B or p85 showed similar stretch-induced phosphorylation of PDGFR-B and increased association with p85. Stretch greatly increased the PDGFR-B associated with p85 following immunoprecipitation with antibodies specific for p85.

### Example 10

### Mechanistic evaluation of stretch-induced VEGF expression.

To determine the mechanism by which stretch increased VEGF mRNA expression, inhibitors of MEK1 (PD98059, 20 uM), classical/novel PKC isoforms (GF109203X, 5 uM), tyrosine phosphorylation (genistein, 20 uM) and PI3 kinase (wortmannin, 100 nM and LY294002, 50 uM) were evaluated. In all experiments 9%/60 cpm cyclic stretch for 3 hours induced VEGF mRNA expression. Inhibition of ERK1/2 utilizing PD98059 had little effect on either basal or stretch-induce expression of VEGF. Similarly, inhibition of PKC classical/novel isoforms utilizing GF109203X did not alter VEGF mRNA expression. In contrast, inhibition of PI3 kinase utilizing either the inhibitor LY294002 or wortmannin resulted in marked inhibition of stretch-induced VEGF mRNA expression without significantly altering basal expression levels. LY294002 and wortmannin inhibited stretched induced VEGF mRNA expression by 85±20% (p=0.039) and 96±25% (p=0.035), respectively. Addition of genistein inhibited stretch-induced VEGF mRNA expression 87±12% (p=0.041), also without altering basal VEGF expression. These results suggest that tyrosine phosphorylation events and activation of PI3 kinase are required for stretched-induced VEGF mRNA expression, whereas activation of classical/novel PKC isoforms and ERK1/2 are not major contributors to this response.

Further confirmation that stretch-induced ERK1/2 activation was not involved in mediating stretch-induced VEGF expression was obtained by assaying ERK1/2 phosphorylation after exposure to the inhibitors described herein. The inhibitor response for stretch-induced ERK1/2 phosphorylation was opposite that observed for stretch-induced VEGF expression. Stretch-induced ERK1/2 phosphorylation was reduced by inhibition of MEK1 (85+10.8%, 88+7.1%, p<0.05) or classical/novel PKC (83+23%, 84+7.1% p<0.05) but relatively unaffected by inhibition of PI3 kinase or tyrosine phosphorylation. Adenovirus infection with dominant negative ERK, wild type active ERK63 or b-galactosidase control had no effect on stretch-induced VEGF expression.

The mechanism of stretch-induced Akt phosphorylation was evaluated using two PI3 kinase inhibitors (LY294002 and wortmannin), the MEK1 inhibitor PD98059 and the tyrosine kinase inhibitor genistein. As observed with stretch-induced VEGF expression, LY294002, wortmannin and genistein inhibited stretch-induced Akt phosphorylation by 119+14% (p<0.001), 119+18% (p<0.001), and 84+14% (p<0.002), respectively, while MEK1 inhibition and classical/novel PKC isoform inhibition had little effect. Basal Akt phosphorylation was also reduced by inhibition of PI3 kinase (p<0.01). The role of PI3 kinase in mediating stretch-induced Akt phosphorylation was confirmed by adenovirus infection with a dominant negative mutant of the p85 subunit of PI3 kinase and a b-galactosidase (bgal) control.

To determine if Akt mediated stretch-induced VEGF expression, adenovirus infection using constitutively active (ca Akt) or dominant negative mutant Akt (mt Akt) was performed. Overexpression of constitutively active Akt did not increase basal or stretch-induced VEGF mRNA expression as compared with b-galactosidase control infected cells. The effective of dominant negative Akt expression was variable and did not demonstrate a statistically significant effect. Further confirmation that PI3 kinase was important in stretch-induced VEGF expression was obtained using adenoviral infection with the dominant negative mutant of the p85 subunit of PI3 kinase (D85) which inhibited stretch-induced VEGF mRNA expression by 130+24.5% (p<0.01) without altering basal VEGF expression.

### Example 11

### Role of PKC-zeta in stretch-induced VEGF expression.

Since the PKC inhibitors evaluated in this study effect novel and classical isoforms of PKC but not atypical isoforms, and since PI3 kinase has been reported to activate the atypical zeta isoform of PKC53 (Akimoto et al. (1996) EMBO J. 15:788-798), the role of PKC-zeta in stretch-induced VEGF expression was evaluated. To determine if PKC-zeta was actually expressed in retinal pericytes, western blot analysis using PKC-z specific antibody was performed. Retinal pericytes clearly expressed PKC-zeta protein and expression was greater than that observed in retinal endothelial cells. Adenovirus mediated overexpression of wild type classical PKC isoform α, novel PKC isoform δ or green fluorescent protein control (GFP) had no effect on either basal or stretch-induced VEGF mRNA expression. In contrast, overexpression of the wild type atypical zeta isoform of PKC further increased stretch-induced VEGF mRNA expression 91+48% (p<0.04) while dominant negative expression of PKC-zeta inhibited stretch-induced VEGF expression by 73+25% (p<0.02) as compared with GFP control. Basal VEGF mRNA expression was not changed. In contrast, adenovirus mediated expression of wild type or dominant negative mutant PKC-zeta did not effect either basal or stretch-induced ERK 1/2 phosphorylation or Akt expression or phosphorylation as compared with GFP control infected cells.

The effect of cyclic stretch on PKC-zeta activity and its relation to PI3 kinase activation was evaluated. PKC-zeta specific activity was increased 2.6+0.7-fold by 15 minutes of 9% cyclic stretch, a response completely inhibited by the PI3 kinase inhibitor wortmannin (p<0.01).

In human fibrosarcoma and renal cell carcinoma cells, Ras can promote VEGF transcription by activating PKC-zeta. To evaluate whether a similar mechanism was involved in stretch-induced VEGF expression, cells underwent adenoviral infection with dominant negative mutant Ras (DN ras) or b-galactocidase control. DN ras did not effect basal or stretch-induced VEGF expression. In contrast, DN Ras inhibited stretch-induced ERK 1 and ERK 2 phosphorylation by 73+14% (p=0.003) and 70+20% (p=0.007), respectively. These data suggest that stretch-induced, PKC-zeta-mediated VEGF expression occurs via a mechanism not predominantly involving Ras or ERK 1/2.

### Example 12

### Materials and Methods

Reagents:

α]-dCTP and [γ32^{P} ]-dATP were obtained from NEN (Boston, Mass.). Plasmaderived horse serum, fibronectin, sodium pyrophosphate, sodium fluoride, sodium orthovanadate, aprotinin, leupeptin, and phenylmethylsulfonyl fluoride were obtained from Sigma (St. Louis, Mo.). Rabbit polyclonal anti-phospho-p44/p42, anti-phospho-Akt, and anti-Akt antibodies were purchased from New England Biolabs (Beverly, Mass). Mouse monoclonal anti-phosphotyrosine antibody (4G10) was obtained from Upstate Biotechnology, Inc (Lake Placid, N.Y.). Rabbit polyclonal anti-ERK1 antibody, anti-human VEGF antibody and anti-rabbit PKC-z antibody were purchased from Santa Cruz biotechnology, Inc. (Santa Cruz, Calif.). Reagents for sodium dodecyl sulfate-polyacrylamide gel electrophoresis (SDS-PAGE) were obtained from Bio-Rad (Richmond, Calif.). Protein-A Sepharose was purchased from Amersham Pharmacia Biotech (Piscataway, N.J.). Phosphatidylinositol (PI) was purchased from Avanti (Alabaster, Ala.). PD98059, genistein, wotmannin, LY294002 and GF109203X were obtained from Calbiochem (La Jolla, Calif.). All other materials were ordered from Fisher Scientific (Pittsburgh, Pa.) and Sigma (St. Louis, Mo.).

Mechanical Stretch

Cells were seeded on 6-well flexible-bottom plates coated with bovine fibronectin and subjected to uniform radial and circumferential strain using a computercontrolled, vacuum stretch apparatus (Flexcer Cell Strain Unit; Flexcel Corp.) with cardiac profile at a frequency of 60 cpm.

³H-Thymidine Incorporation

Confluent cells were placed in 1% calf serum for 24 hours and then subjected to 9%/60 cpm cyclic stretch for 24 hrs. During the last 12 hrs, VEGF (25 ng/ml final). VEGF neutralizing antibody (10 μg/ml was added 30 minutes prior to stretch onset. ³H-thymidine (0.5 μCi/ml)was added during the last 6 hours.

In Vivo Studies

12 week-old male spontaneously hypertensive rats (SHR) and weight-matched Wistar-Kyoto (WKY) control animals were used. Systolic and diastolic blood pressures were measured from each animal by tail cuff. Animals were treated with or without 100 mg/kg/day captopril or 10 mg/kg/day candesartan cilexetil for one week. Drugs were administered in drinking water. Blood measurements were repeated after therapy and the retinas individually isolated.

Multiplex RT-PCR

Primers for VEGF and RRRPPO were derived per Srivastava et al. (Srivastava RK et al (1998) J Mol Endocrinol.21:335-362) KDR (5′- TGG CTC ACA GGC AAC ATC, 3′-CTT CCT TCC TCA CCC TTC G), Flt-I (5′-CTG ACT CTC GGA CCC CTG, 3′-TGG TGC ATG GTC CTG TTG). RNA was isolated from individual retinas and RNA reverse transcribed at 42° C. using random hexamer primers. PCR amplification was carried out using a 55° C. annealing temperature. The samples were separated on a 6% nondenaturing polyacrylamide gel. The gel was dried and analyzed by PhosphorImager. Signal intensity was normalized using rat ribosomal phosphoprotein P0 (RRRPP0) as an internal standard. Band identity was confirmed by monoplex and multiplex reactions, southern blot analysis and bidirectional DNA sequencing.

Cell culture: Primary cultures of bovine retinal pericytes (BRPC) were isolated by homogenization and a series of filtration steps. BRPC were cultured in DMEM containing 5.5 mM glucose and 20% FBS. The cells were maintained in 5% CO2 at 37oC and media were changed every three days. Cells were characterized for their homogeneity by immunoreactivity with monoclonal antibody 3G5. Cells were plated at a density of 2×104 cells/cm2 and passaged when confluent. The media were changed every three days and only cells from passages 2-5 were used for experiments.

Recombinant Adenoviruses: cDNA of constitutive active Akt (ca Akt, Gag protein fused to N-terminal of wild type Akt) was constructed as described.61 cDNA of dominant negative Akt (mt Akt) was constructed by substituting Thr-308 to Ala and Ser-473 to Ala as previously described. cDNA of ERK (extracellular signal-regulated kinase) was constructed as previously described. cDNA of dominant negative mutant ERK (mt ERK)was constructed by substituting Lys-52 to Arg in the ATP-binding site as previously described. cDNA of dominant negative KRas (DNRas, substituted Ser-17 to Asn) was kindly provided by Dr.Takai (Osaka University). cDNA of Dp85 was kindly provided by Dr. Kasuga (Kobe University). cDNAs of PKC β, δ and zeta were kindly provided by Dr. Douglas Kirk Ways (Lilly Laboratory, Indianapolis, Ind.). cDNA of dominant negative PKC-zeta (mt PKC-z) substituting Lys-273 to Trp in the ATPbinding site was constructed as previously described (Uberall et al. (1999) J. Cell Biol. 144, 413-425). The recombinant adenoviruses were constructed by homologous recombination between the parental virus genome and the expression cosmid cassette or shuttle vector as previously described. Adenovirus were applied at a concentration of 1×10⁸ plaque-forming units/ml, and adenovirus with the same parental genome carrying LacZ gene or enhanced green fluorescein protein gene (EGFP, Clonetech, Palo Alto, Calif.) were used as controls. Expression of each recombinant protein was confirmed by Western blot analysis, and expression was increased approximately 10-fold with all constructs as compared to cells infected with control adenovirus.

Mechanical Stretch: For pericyte experiments, cells were plated on 6-well flexiblebottom culture plates coated with collagen (Flexcell Corp.,Mckeepsport, Pa.). After 2 days, media were changed to DMEM containing 1% calf serum and the cells incubated overnight. Cells were then subjected to uniform radial and circumferential strain in 5% CO2 at 37° C. using a computer-controlled, vacuum stretch apparatus (Flexcer Cell Strain Unit; Flexcel Corp.). A physiologic stretch frequency of 60 cpm and 3-20% prolongation of elastomer bottomed plates were used as previously described.

RNA Extraction: RNA was extracted using the guanidinium thiocynate method. RNA purity was determined by the ratio of optical density (OD) measured at 260 & 280 nm and RNA quantity was estimated using OD measured at 260 nm.

Northern Blot Analysis: Northern blot analysis was performed on 15 μg total RNA per lane after 1% agarose-2M formaldehyde gel electrophoresis and subsequent capillary transfer to Biodyne nylon membranes (Pall BioSupport, East Hills, N.Y.). Membranes underwent ultraviolet crosslinking using a UV Stratalinker 2400 (STRATAGENE, La Jolla, Calif.). Radioactive probes were generated using Amersham Megaprime labeling kits (Buckinghamshire, England) and 32PdCTP (NEN Life Science Products Inc., Boston, Mass.). Blots were pre-hybridized, hybridized and washed 4 times in 0.5×SSC, 5% SDS at 65oC for 1 hour in a rotating hybridization oven (Robbins Scientific Corporation, Sunnyvale, Calif.). All signals were analyzed using a computing PhosphorImager with ImageQuant software analysis (Molecular Dynamics, Sunnyvale, Calif.). The signal for each sample was normalized by re-probing the same blot using 36B4 cDNA control probe.

VEGF mRNA Half-life Analysis: BRPC were cultured as indicated above and exposed to 9%/60 cpm mechanical stretch for 4 hours. Actinomycin D (5μg/ml) was added and RNA isolated 0, 2 and 4 hours later. Northern blot analysis of these samples was performed and quantitated as described above.

VEGF and PKC-zeta protein detection: BRPC were washed with cold PBS and lysed in 1X Laemmli buffer (50 mmol/Tris, pH6.8, 2% SDS, 10% glycerol) containing protease inhibitors [10 mmol/l sodium pyrophosphate, 100 mmol/l sodium fluoride (NaF), 1 mmol/l sodium orthovanadate (Na3VO4), 1 μg/ml aprotinin, 1 μg/ml leupeptin, and 2 mmol/l phenylmethylsulfonyl fluoride (PMSF)]. Protein concentrations were determined with Bio-Rad protein assay (Bio-Rad Laboratories, Hercules, Calif.) Total cell lysate (30 ug) was subjected to SDS-polyacrylamide gels (SDS-PAGE) under reducing conditions, and proteins were transferred to nitrocellulose membrane (Bio-Rad, Hercules, Calif.). The blots were incubated with primary antibodies followed by incubation with horseradish peroxidase-conjugated secondary antibody (Amersham, Piscataway, N.J.). Visualization was performed using Amersham Enhanced Chemiluminescence detection system (ECL) per manufacturer's instructions.

ERK1/2 and Akt phosphorylation: Cells were washed with cold PBS and lysed in 1X Laemmli buffer containing protease inhibitors as described above. Cell lysates were heated to 95 oC for 2 min and equal volume of lysates were subjected to SDS-PAGE under reducing conditions. The blots were incubated with anti-phosphospecific ERK1(p44)/ERK2(p42) or anti-phosphospecific Akt antibody (New England Biolabs, Beverly, Ma.). Lane loading differences were normalized by reblotting with non-phosphorylation-specific (total) anti ERK1 antibody (Santa Cruz Biological) or anti-total Akt antibody(New England Biolabs).

PI3-Kinase Assay: PI3-kinase activity was measured by in vitro phosphorylation of PI. Cells were lysed in ice-cold lysis buffer containing 50 mM Hepes, pH 7.5, 137 mM NaCl, 1 mM MgCl2, 1 mM CaCl2, 2 mM Na3VO4, 10 mM NaF, 2 mM EDTA, 1% Nonidet P-40, 10% glycerol, 1 mM PMSF, 2μg/ml aprotinin, 5 μg/ml leupeptin, and 1 μg/ml pepstatin. Insoluble material was removed by centrifugation at 15,000 g for 10 min at 4 ° C. PI 3-kinase was immunoprecipitated from aliquots of the supernatant with antiphosphotyrosine antibodies. After washing, the pellets were resuspended in 50 μl of 10 mM Tris (pH 7.5), 100 mM NaCl, and 1 mM EDTA. 10 μl of 100 mM MgCl2 and 10 μl of PI (2 μg/μl) sonicated in 10 mM Tris (pH 7.5) with 1 mM EGTA was added to each pellet. The PI 3-kinase reaction was initiated by the addition of 5 μl of 0.5 mM ATP containing 30 μCi of [g32P]-ATP. After 10 min at room temperature with constant shaking, the reaction was stopped by the addition of 20 μl of 8 N HCl and 160 μl of chloroform/methanol (1:1). The samples were centrifuged, and the organic phase was removed and applied to silica gel TLC plates developing in CHC13:CH3OH:H2O:NH4OH (60:47:11:2). The radioactive spots were quantitated by PhosphorImager (Molecular Dynamics, Sunnyvale, Calif.).

PKC-zeta Activity: PKC-z activity was measured as described int eh art. Briefly, cells were lysed in 0.5% Triton X-100, 50 mM Tris-HCl(pH 7.5), 10% glycerol, 2 mM DTT, 5 mM EDTA, 5 mM EGTA, 20 mM NaF, 2 mM Na3VO4 and 2 mM PMSF. The lysates were subjected to immunoprecipitation with polyclonal antibodies against PKC-zeta. The immunocomplexes were incubated at 30° C. for 15 min in 50 μl of kinase assay mixture containing 35 mM Tris-HCl(pH7.5), 10 mM MgCl2, 0.5 mM EGTA, 0.1 mM CaCl2, 40 μM ATP, 0.5 μCi of [g32P] ATP and 30 μM PKC-e pseudosubstrate peptide (Biosource, Camarillo, Calif.). Aliquots of reaction mixtures were spotted on p81 filter paper (Whatman, Maidstone, UK) and washed with 75 mM phosphoric acid. The radioactivity incorporated into phosphorylated substrate proteins was quantitated by scintillation counter.

Statistical Analysis: All experiments were repeated at least three times unless otherwise indicated. Results are expressed as mean ± standard deviation. Statistical analysis employed Student's t-test or analysis of variance to compare quantitative data populations with normal distributions and equal variance. Data were analyzed using the Mann-Whitney rank sum test or the Kruskal-Wallis test for populations with non-normal distributions or unequal variance. A Pvalue of <0.05 was considered statistically significant.

Other embodiments are within the following claims.

We claim:

1. A method of treating hypertension or a hypertension-related disorder in a subject, comprising:
identifying a subject in need of treatment for hypertension or a hypertension-related disorder; and
administering to a cell or tissue of the subject an agent that inhibits a component of the VEGF-KDR signaling pathway.

2. The method of claim 1, wherein the agent decreases the expression, level or activity of VEGF.

3. The method of claim 2, wherein the agent is selected from the group of: a VEGF binding protein that inhibits VEGF binding to KDR; an antibody to VEGF that inhibits VEGF activity; a mutated VEGF or fragment thereof that inhibits VEGF signaling; a VEGF nucleic acid molecule that inhibits expression of VEGF; and a small molecule that inhibits transcription or activity of VEGF.

4. The method of claim 1, wherein the agent decreases the expression, level or activity of KDR.

5. The method of claim 4, wherein the agent is selected from the group of: a KDR-binding protein that inhibits VEGF binding to KDR; an antibody to KDR that inhibits KDR activity; a mutated KDR or fragment thereof that inhibits KDR signaling; a KDR nucleic acid molecule that inhibits expression of KDR; and a small molecule that inhibits transcription or activity of KDR.

6. The method of claim 1, wherein the agent decreases the expression, level or activity of PI3 kinase.

7. The method of claim 6, wherein the agent is selected from the group of: a PI3 kinase binding protein that inhibits PI3 kinase activity; an antibody to PI3 kinase that inhibits PI3 kinase activity; a mutated PI3 kinase or fragment thereof that inhibits PI3 kinase activity; a PI3 kinase nucleic acid molecule that inhibits expression of PI3 kinase; and a small molecule that inhibits transcription or activity of PI3 kinase.

8. The method of claim 7, wherein the agent is LY294002.

9. The method of claim 7, wherein the agent is wortmannin.

10. The method of claim 1, wherein the agent decreases PKC-zeta expression, levels or activity.

11. The method of claim 10, wherein the agent is selected from the group of: a PKC-zeta binding protein that inhibits PKC-zeta activity; an antibody to PKC-zeta that inhibits PKC-zeta activity; a mutated PKC-zeta or fragment thereof that inhibits PKC-zeta activity; a PKC-zeta nucleic acid molecule that inhibits expression of PKC-zeta; and a small molecule that inhibits transcription or activity of PKC-zeta.

12. The method of claim 1, wherein the hypertension related disorder is retinopathy.

13. The method of claim 1, wherein the cell or tissue is a retinal cell or tissue.

14. A method of screening for a compound that decreases hypertension or a hypertension-related disorder, comprising:
providing a cell, tissue, or subject;
contacting the cell, tissue, or subject with a test compound; and
determining whether the test compound inhibits a component of the VEGF-KDR signaling pathway.

15. The method of claim 14, further comprising subjecting the cell, tissue or subject to a mechanical stress.

16. The method of claim 14, wherein the cell is an endothelial cell

17. The method of claim 14, wherein the cell is a bovine retinal endothelial cell (BREC) or bovine retinal pericyte (BRPC).

18. A method of determining if a subject is at risk for hypertension or a hypertension related disorder, comprising detecting the misexpression or mutation of a gene involved in VEGF-KDR signaling.

19. The method of claim 18, wherein the gene involved in VEGF-KDR signaling is selected from the group of: VEGF, KDR, PI3 kinase, and PKC-zeta.

20. The method of claim 18, wherein the hypertension related disorder is retinopathy.
